(19) United States
(12) Patent Application Publication
(10) Pub. No.: US 2008/0064884 A1
(43) Pub. Date: Mar. 13, 2008

(54) Novel Process 1

(75) Inventors: Debra Ainge, Loughborough (GB); Philip Cornwall, Bangalore (IN); Duncan Michael Gill, Loughborough (GB); Vinod Kumar, Bangalore (IN); Luis Manuel Vaz, Loughborough (GB); Philip O'Keefe, Loughborough (GB); Rhona Sinclair, Loughborough (GB); Edward Laurence Way, Loughborough (GB)

Correspondence Address: FISH & RICHARDSON P.C., P.O BOX 1022, MINNEAPOLIS, MN, 55440-1022, US

(73) Assignee: AstraZeneca AB, SE-151 85 Sodertalje (SE)

(21) Appl. No.: 11/743,773

(22) Filed: May 3, 2007

Related U.S. Application Data
(60) Provisional application No. 60/799,574, filed on May 10, 2006.

(51) Int. Cl. C07D 303/36; C07D 317/28; C07D 317/18

(52) U.S. Cl. 549/445; 549/551; 564/218; 564/443; 568/587

(57) ABSTRACT

The present invention relates to a novel process for the preparation of compounds of formula (I)

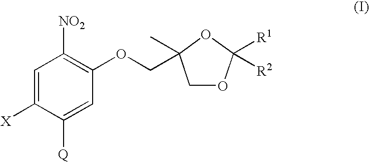

wherein X, Q, R¹ and R² are as defined in the specification, the compounds being useful in the preparation of therapeutic agents. The invention further relates to novel intermediates useful in the preparation of the therapeutic agents.

The present invention relates to novel processes for the preparation of intermediate compounds which can be used to prepare therapeutic agents. The present invention also relates to novel intermediate compounds which can be used to prepare therapeutic agents.

Chemokines play an important role in immune and inflammatory responses in various diseases and disorders, including asthma and allergic diseases, as well as autoimmune pathologies such as rheumatoid arthritis and atherosclerosis. Studies have demonstrated that the actions of chemokines are mediated by subfamilies of G protein-coupled receptors, among which are the receptors designated CCR1, CCR2, CCR2A, CCR2B, CCR3, CCR4, CCR5, CCR6, CCR7, CCR8, CCR9, CCR10 and CCR11 (for the C-C family); CXCR1, CXCR2, CXCR3, CXCR4 and CXCR5 (for the C-X-C family) and CX₃CR1 and the C-X₃-C family. These receptors represent good targets for drug development since agents which modulate these receptors would be useful in the treatment of disorders and diseases such as those mentioned above.

WO01/98273 discloses a series of compounds having a structure (IA) shown below, where R^{b} is a phenyl group (which may be substituted) and where R^{b} represents a suitable substituent and n is typically 0, 1 or 2.

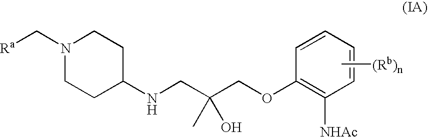

WO03/051839 discloses the CCR1 antagonist N-{2-[((2S)-3-{[1-(4-chlorobenzyl)piperidin-4-yl]amino}-2-hydroxy-2-methylpropyl)oxy]-4hydroxyphenyl}acetamide. A related compound, N-{5-Chloro-2-[((2S)-3-{[1-(4-chlorobenzyl)piperidin-4-yl]amino}-2-hydroxy-2-methylpropyl)oxy]-4-hydroxyphenyl}acetamide has also been shown to antagonise CCR1 activity.

Methods of synthesising compounds of the type described above typically involve alkylation of a protected acetamidophenol derivative (2) with an expoxide derivative e.g. [2-methyloxiranyl]methyl-3-nitrobenzene sulfonate (3) (also known as methylglycidyl nosylate) to give an epoxy ether derivative (4) e.g. as shown in step (i) of scheme 1 below. Reaction of the epoxide product (4) with a piperidine amine (5) as shown in step (ii) of scheme 1 (and deprotection of any protected substituent groups) can give rise to the target pharmaceutical compound (1A).

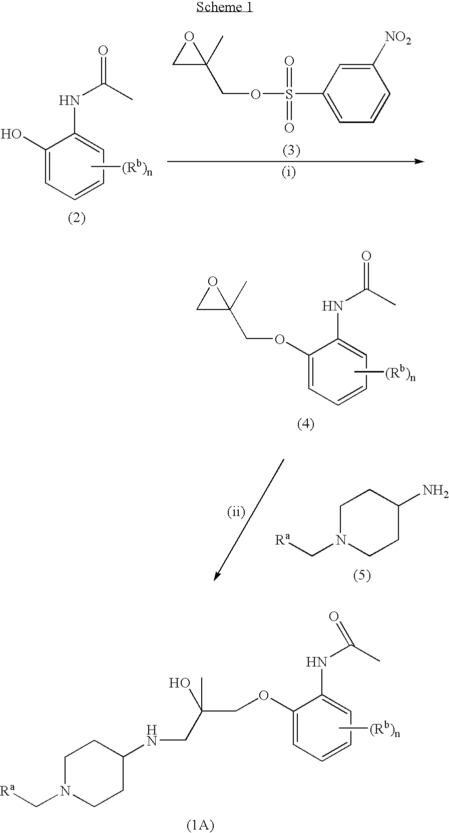

Whilst acceptable as a method to prepare target compounds in quantities of up to five kilograms, such routes are not considered suitable for further scale-up. One reason for this is the safety issues surrounding the transport and handling of the glycidyl nosylate (3), which has been found to have potentially dangerous thermal properties. Furthermore, known methods for the synthesis and purification of the glycidyl nosylate (3) can give rise to variable yields and significant levels of by-products.

In view of the above, it would be advantageous to find new methods of synthesising compounds of formula (A).

The present invention provides a process of preparing a compound of formula (I) or a salt thereof:

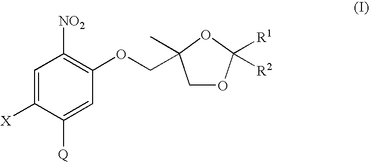

wherein Q is OH or OP where P is an alcohol-protecting group or Q is fluorine or chlorine,
X is hydrogen or chlorine,
and R¹ and R² together with the carbon atom to which both are attached form a 1,2 diol protecting group,
which process comprises reacting a compound of formula (II) or a salt thereof

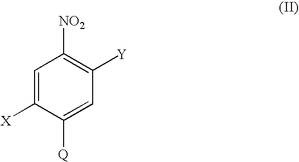

wherein Q and X are as defined in formula (I), and Y is chlorine or fluorine,
with a compound of formula (III) or a salt thereof

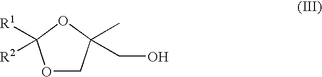

wherein R¹ and R² are as defined in formula (I),
in the presence of a base.

In one embodiment of the process of the invention, Y in formula (II) is fluorine.

In a further embodiment of the process of the invention, Q in formula (I) and formula (II) is OH or OP.

In a further embodiment of the process of the invention, Q in formula (I) and formula (II) is fluorine.

In a further embodiment of the process of the invention, X in formula (I) and formula (II) is hydrogen.

In a further embodiment of the process of the invention, X in formula (I) and formula (II) is chlorine.

In a further embodiment of the process of the invention, X in formula (I) and formula (II) is hydrogen or chlorine, Q is OH or OP, and Y is fluorine.

In a further embodiment of the process of the invention, X in formula (I) and formula (II) is hydrogen or chlorine, Q is fluorine and Y is fluorine.

In a further embodiment of the process of the invention, X in formula (I) and formula (II) is hydrogen or chlorine, Q is chlorine and Y is chlorine.

In a further embodiment of the process of the invention, X in formula (I) and formula (II) is hydrogen or chlorine, Q is chlorine and Y is fluorine.

R¹ and R² together with the carbon atom to which both are attached form a 1,2 diol-protecting group. The 1,2 diol protecting group can be chosen such that its removal can provide the corresponding 1,2 diol. 1,2 diol-protecting groups and methods for their removal are well known in the art. For example, methods to effect deprotection of 1,2 diol-protecting groups are outlined in ‘Protective Groups in Organic synthesis’, 3rd edition, T. W. Greene and P. G. M. Wutz, Wiley-Interscience (1999).

R¹ and R² may, for example, each independently represent hydrogen or C₁-C₆ alkyl (e.g. methyl or ethyl), or R¹ and R², together with the carbon atom to which they are both attached may form a C₄-C₇ cycloalkyl ring, more preferably a cyclopentyl or cyclohexyl ring or R¹ and R² together with the carbon atom to which they are both attached form a C₅-C₇ cycloalkyl ring; or R¹ is hydrogen or methyl and R² is phenyl or 4-methoxyphenyl. Alternatively, R¹ may be hydrogen or methyl with R² being phenyl. Alternatively still, R¹ may be hydrogen or methyl with R² being 4-methoxyphenyl.

In a preferred embodiment, R¹ and R² are each methyl.

Unless otherwise indicated, the term ‘alkyl’ when used alone or in combination, refers to a straight chain or branched chain alkyl moiety. A C₁-C₆ alkyl group has from one to six carbon atoms including methyl, ethyl, n-propyl, isopropyl, tert-butyl, n-pentyl, n-hexyl and the like.

In this specification, unless stated otherwise, the term “cycloalkyl” refers to an optionally substituted, partially or completely saturated monocyclic, bicyclic or bridged hydrocarbon ring system. The term “C₁₋₆cycloalkyl” may be, but is not limited to cyclopropyl, cyclobutyl, cyclopentyl or cyclohexyl.

The process of the present invention is typically carried out in the presence of a base, typically alkali metal bases such as, but not limited to, potassium hydroxide, sodium hydroxide, sodium hydride, potassium hydride, potassium tert-butoxide, potassium tert-pentylate, potassium-3,7-dimethyl-3-octylate.

The process of the present invention is carried out in a suitable solvent, for example a hydrocarbon, nitrile, polar aprotic or ether solvent. Suitable solvents include tetrahydrofuran, 2-methyl tetrahydrofuran, diethyl ether, di-isopropyl ether, acetonitrile, butyronitrile, N-methyl pyrrolidinone, dimethylacetamide, dimethyl formamide, dimethyl sulfoxide, toluene and xylenes, and combinations thereof. In one embodiment of the invention, the solvent is toluene or a mixture of toluene and N-methyl pyrrolidinone.

Typically, the process is carried out at temperatures between −78° C. and 120° C., more preferably between −10° C. and 70° C. When Q is OH, the reaction is preferably carried out above 20° C. temperature, and when Q is OP or halogen, the reaction is preferably carried out at or below 20° C. temperature.

Compounds of formula (I) are capable of existing in stereoisomeric forms, and it will be understood that the invention encompasses synthesis of all optical isomers of the compounds of formula (I) and mixtures thereof including racemates. A specific enantiomer of a compound of formula (I) can be prepared by using a corresponding specific enantiomer of a compound of formula (III). Reference to specific enantiomers of compounds of formulas (I) or (III) refers to the stereochemistry at the centre marked * below

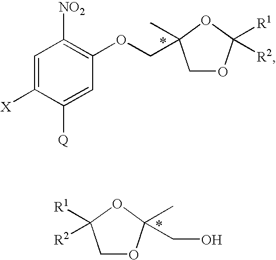

For example, use of the R-enantiomer of a compound of formula (III) in the process of the invention can give the corresponding R-enantiomer of a compound of formula (I). Alternatively, a required enantiomer of formula (I), which racemic mixture may be prepared from a racemic compound of formula (III). Techniques for separation of enantiomers from racemic mixtures are well known in the art. Alternatively, the compound of formula (I) can be converted into a racemic epoxide (see later) which epoxide can then be transformed into an enantiomerically enriched diol via enzymatic de-racemisation using method such as those described in Tetrahedron Asymmetry, 2006, 17, 402.

Preferably, when R¹ and R² are methyl, the process of the present invention is used for the preparation of a compound corresponding to the R-isomer of formula (I).

Accordingly, in one embodiment the process of the present invention comprises reacting a compound of formula (II) with a compound corresponding to the R-isomer of formula (III) where R¹ and R² are both methyl. Alternatively, the R-isomer of formula (I) may, for example, be obtained from a racemic mixture of compound of formula (I).

Group Q in formula (I) and formula (II) may be OH or OP where P is an alcohol-protecting group.

The alcohol-protecting group P may in general be chosen from any of the groups described in the literature or known to the skilled chemist as appropriate for the protection of the group in question and may be introduced by conventional methods. The protecting group may be removed by any convenient method as described in the literature or known to the skilled chemist as appropriate for the removal of the protecting group in question, such methods being chosen so as to effect removal of the protecting group with minimum disturbance of groups elsewhere in the molecule. The protection and deprotection of hydroxy functional groups is well known in the art, and is described, for example, in ‘Protective Groups in Organic Chemistry’, edited by J. W. F. McOmie, Plenum Press (1973) and ‘Protective Groups in Organic Synthesis’, 3rd edition, T. W. Greene and P. G. M. Wutz, Wiley-Interscience (1999). Specific examples of protecting groups are given below for the sake of convenience, in which “lower”, as in, for example, lower alkyl, signifies that the group to which it is applied preferably has 1-4 carbon atoms. It will be understood that these examples are not exhaustive. Where specific examples of methods for the removal of protecting groups are given below these are similarly not exhaustive. The use of protecting groups and methods of deprotection not specifically mentioned are, of course, within the scope of the invention.

Examples of alcohol-protecting groups that may be used in the present invention include lower alkyl groups (for example tert-butyl), lower alkenyl groups (for example allyl); lower alkanoyl groups (for example acetyl); lower alkoxycarbonyl groups (for example tert-butoxycarbonyl); lower alkenyloxycarbonyl groups (for example allyloxycarbonyl); aryl-lower alkoxycarbonyl groups (for example benzyloxycarbonyl, 4-methoxybenzylocycarbonyl, 2-nitrobenzyloxycarbonyl and 4-nitrobenzyloxycarbonyl); tri(lower alkyl)silyl (for example trimethylsilyl and tert-butyldimethylsilyl) and aryl-lower alkyl (for example benzyl) groups. On one embodiment of the invention P is a lower alkanoyl groups such as acetyl.

Typical protecting groups that may be used in the present invention include alkyl, allyl, acyl, benzyl, benzhydryl, trityl, or trialkylsilyl protecting groups. P may for example be methyl, ethyl, isopropyl, benzyl, p-methoxybenzyl or trityl; an alkoxyalkyl ether such as, but not limited to methoxymethyl; benzyl; or tetrahydropyranyl. The group OP may be an ester such as, but not limited to, acetate (i.e. P being acetyl) and benzoate. The group OP may be a silyl ether with P being, but not limited to, trimethylsilyl, triethylsilyl, tri-isopropylsilyl, tert-butyldimethylsilyl or tert-butyldiphenylsilyl. In one embodiment P is selected from C₁-C₄ alkyl groups, C₁-C₄ alkenyl groups, C₁-C₄ alkanoyl groups, C₁-C₄ alkoxycarbonyl groups, C₁-C₄ alkenyloxycarbonyl groups, aryl-C₁-C₄ alkoxycarbonyl groups, tri(C₁-C₄ alkyl)silyl and aryl-C₁-C₄ alkyl groups.

Compounds of formula (I), (II) and (III) may be in free base form or in salt form. The use of both free forms and salt forms are within the scope of the present invention. Salts may typically exist when Q in (I) and (II) is OH. Examples of salt forms include a base salt such as an alkali metal salt, for example lithium, sodium or potassium, or an alkaline earth metal salt, for example calcium or magnesium.

Compounds of formula (I) can be converted to compounds of formula (IA) as described later in this application.

The SnAr process chemistry of the present invention is considered to give rise to a number of advantages. For example, the process of the present invention can be carried out using only a slight excess of a compound of formula (II). The process of the present invention can be volume efficient. Furthermore, the process of the invention allows for near stoichiometric quantities of compound of formula (II) and base. The SnAr approach of the present invention is simple to carry out, negating the need for metal catalysis or hazardous reagents. In particular, the process may be carried out without the use of potential genotoxic alkylating agents (e.g. chlorohydrins and sulfonate esters). The SnAr approach can also be carried out using cheap, readily available bases (such as potassium tert-butoxide). The process of the present invention can be operated in hydrocarbon, nitrile and ether solvents and may not necessarily require high boiling dipolar aprotics such as DMF, DMSO and NMP. The SnAr approach of the present invention may also give rise to high yields and low levels of impurities. The SnAr approach also allows for relatively quick reactions.

Compounds of formula (I) in which Q is OH or OP represent particularly suitable intermediates for synthesising compounds of formula (IA) in which an OH group is present para to the acetamide of the right-hand phenyl group [when viewing formula (IA) as set out in scheme 1], as set out in schemes 3 below.

Compounds of formula (I) in which Q is OH or OP can be prepared from compounds of formula (II) in which Q is OH or OP (in the case of OP, removal of the protecting group P being required at some stage during the synthesis of the final product of formula (IA)). However, when Q in formula (II) is OH, the process of the present invention can surprisingly be carried out without a protecting group to prepare a compound of formula (I) in which Q is OH. This can give rise to efficiency gains be negating the need for protection and deprotection steps.

However, it is possible to introduce the OH group into a compound of formula (I) subsequent to the coupling of a compound of formula (II) with a compound of formula (III). This can be achieved by firstly preparing a compound of formula (I)′

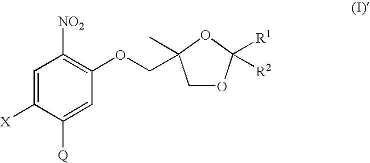

wherein Q is fluorine or chlorine, and X, R¹ and R² are as hereinbefore defined, by reacting a compound of formula (II)′

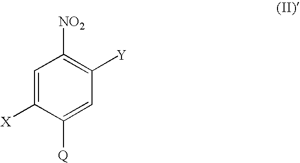

wherein Q is fluorine or chlorine and X and Y are as hereinbefore defined, with a compound of formula (III) or a salt thereof

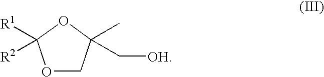

Q and Y in formula (I)′ and formula (II)′ may each independently be chlorine or fluorine. For example, both Q and Y may be fluorine. Alternatively, both Q and Y may be chlorine. In an embodiment of the present invention, X in formula (I)′ and formula (II)′ is hydrogen, R¹ and R² are each methyl, and Q and Y are each fluorine. In a further embodiment of the present invention, X in formula (I)′ and formula (II)′ is hydrogen, R¹ and R² are each methyl, and Q and Y are each chlorine.

The present inventors have found that the SnAr reaction resulting from reacting a compound of formula (II)′ with a compound of formula (III) is surprisingly regioselective for substitution of the halogen at position Y. This regioselectivity can surprisingly be enhanced by employing sub-ambient reaction temperatures (e.g. below 20° C.). The regioselectivity can also surprisingly be enhanced by carrying out the reaction in non-polar solvents. A preferable solvent is toluene. One embodiment relates to the process, wherein Q and Y are each chlorine or Q and Y are each fluorine, and the reaction is carried out in a non-polar solvent such as toluene.

The resulting compound can then undergo a second SnAr reaction in which the fluorine or chlorine in position Q of formula (I)′ is substituted with OH or OP (where P is an alcohol protecting group as defined hereinbefore). This can be achieved, for example, as set out in scheme 2 below, where R is either hydrogen or a protecting group.

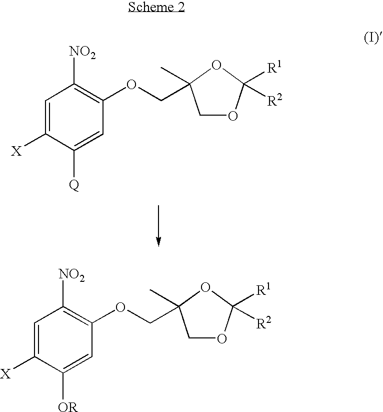

In scheme 2, Q can be replaced with OH using hydroxide sources such as, but not limited to potassium hydroxide, sodium hydroxide and Triton B, or a combination thereof. Such reactions can be carried out at temperatures typically between 40-130° C. in solvents such as hydrocarbons (toluene), polar aprotic (dimethylsulfoxide and N-methyl pyrrolidinone) and alcohols (tert-butanol). Q can be replaced with OH using a phase transfer catalyst, such as Triton B and an aqueous base, such as potassium hydroxide and sodium hydroxide and a non polar solvent, such as toluene. In addition, OH can be introduced using reagents, that upon work-up liberate a free OH group. Such reagents include, but are not limited to, 2-butyn-1-ol (synthetic communications, 32 (9), 1401, 2002) and 2-(methylsulfonyl)ethanol (Tetrahedron Letters, 43, 3585, 2002).

In scheme 2, Q can be replaced with OR by reaction with the corresponding alcohol ROH in the presence of a base, typically alkali metal bases such as, but not limited to, potassium hydroxide, sodium hydroxide, sodium hydride, potassium hydride, potassium tert-butoxide, potassium tert-pentylate, potassium-3,7-dimethyl-3-octylate, butyl lithium, lithium di-isopropylamide, lithium hexamethyldisilazane or combinations thereof, more preferably sterically hindered alkali metal alkoxides such as, but not limited to potassium tert-butoxide, potassium tert-pentylate and potassium-3,7-dimethyl-3-octylate. Such a reaction is carried out in a suitable solvent, for example solvents such as, but not limited to ethers (tetrahydrofuran, 2-methyl tetrahydrofuran, diethyl ether and di-isopropyl ether), nitriles (acetonitrile and butyronitrile), polar aprotic solvents (N-methyl pyrrolidinone, dimethylacetamide and dimethyl formamide) and hydrocarbons (toluene and xylenes) and combinations thereof, more preferably toluene or a mixture of toluene and N-methyl pyrrolidinone. Typically, such a reaction is carried out at temperatures between −78 ° C. and 120° C., e.g. between −78° C. and 25° C.

compounds of formula (1) where X is hydrogen or chlorine, and Q is OH or OP (where P is an alcohol protecting group as defined hereinbefore) can be converted into epoxide intermediates as set out in scheme 3 below.

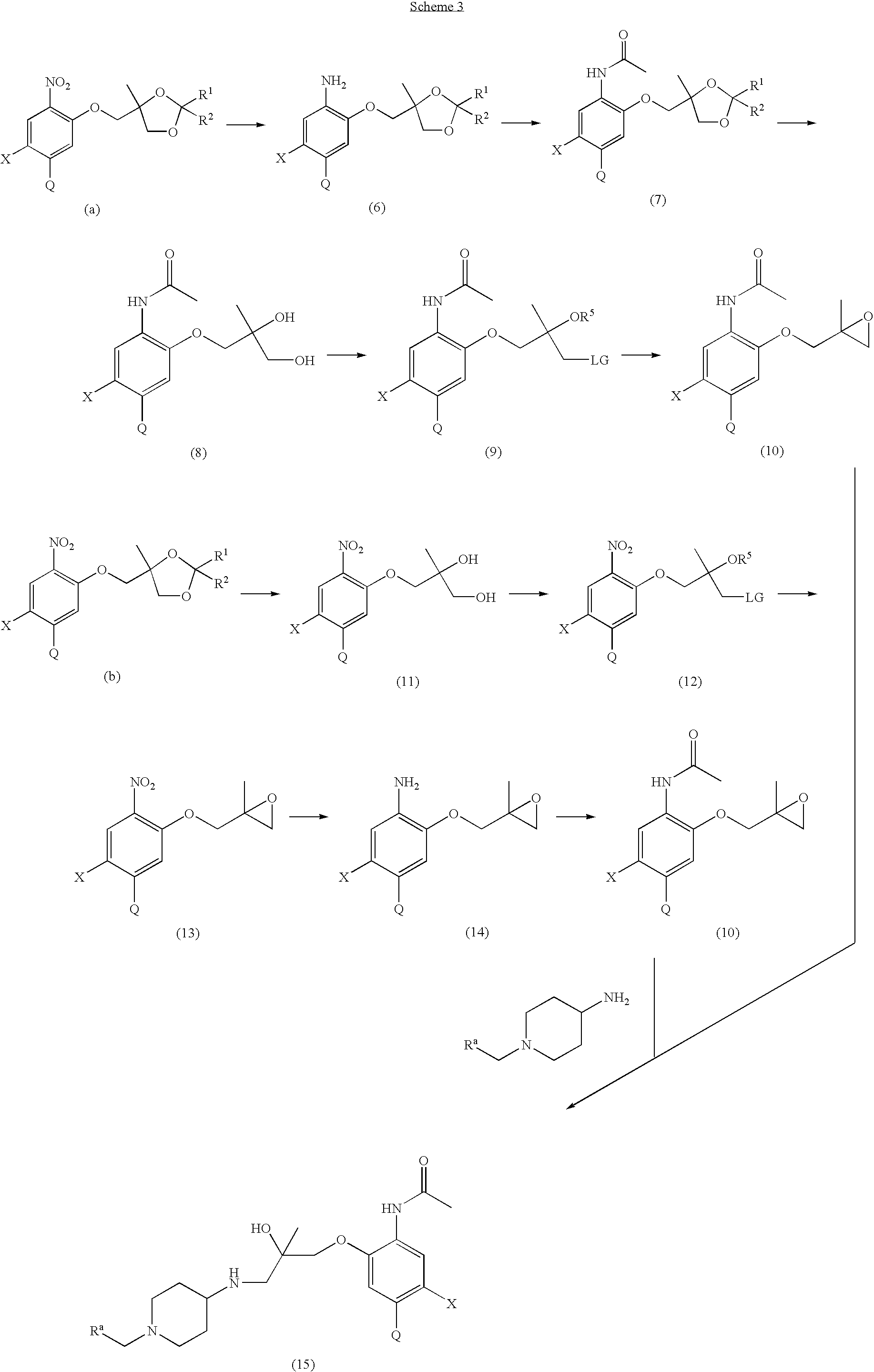

In route (a) of scheme 3, compounds (6) are produced by reduction of the nitro group. This can be carried out using standard reduction techniques, for example using catalytic hydrogenation or sodium dithionite. Compounds (6) can be converted to compounds (7) using standard acetylation techniques (e.g. by reacting with acetic anhydride or acetyl chloride).

Compounds (7) can be converted to compounds (10) using standard techniques, for example removal of the diol protecting group to give the 1,2 diol (8), followed by activation of the primary alcohol, and base mediated ring closure. For example, where R¹ and R² are alkyl groups e.g. methyl, the diol protecting group can be removed using standard techniques, such as, but not limited to, acid catalysed hydrolysis using acids such as HCl, acetic acid, para-toluene sulfonic acid or ion exchange resins such as Dowex 50.

The primary alcohol of 1,2 diol (8) can be activated to form a leaving group (LG), for example, as a sulfonate ester, such as, but not limited to, tosylate, nosylate and mesylate. These are prepared using standard techniques (tosyl, nosyl or mesyl chloride plus base respectively). Alternatively, the primary alcohol can be converted to the bromide using HBr or acetyl bromide in acetic acid to give the bromo acetoxy derivative [i.e. R³=CH₃C(O)—), and LG=Br]. Upon treatment with base, the bromo acetoxy derivative forms the bromohydrin (i.e. R³=H).

The activated diols can be transformed to the epoxides (e.g. 10 13) upon treatment with a base using standard techniques. Suitable alkali metal bases include, but are not limited to, potassium carbonate, sodium hydroxide, potassium hydroxide, sodium hydride, sodium methoxide and sodium ethoxide.

In route (b) of scheme 3, the epoxide (13) is formed first (which can be achieved using the analogous methods to those set out for compounds (10) above. The nitro group is then reduced to produce compounds (14) with the subsequent acetylation to compounds (10).

One embodiment relates to a process for the chemoselective reduction of an aromatic nitro group in the presence of an epoxide. Another embodiment relates to a process for the chemoselective reduction of an aromatic nitro group in the presence of an epoxide using a platinum catalyst. Yet a further embodiment relates to a process for the reduction of an aromatic nitro group and in situ acetylation in the presence of an epoxide using a platinum catalyst. Yet another embodiment relates to a process for the reduction of an aromatic nitro group and in situ acetylation in the presence of an epoxide using a platinum catalyst to give an aromatic amide. Another embodiment relates to a process for the chemoselective reduction and in situ acetylation of compounds (13), using a platinum catalyst. One embodiment relates to a process for the chemoselective reduction and in situ acetylation of compounds (13), using a platinum catalyst, to give compounds of the formula (10). A process for the chemoselective reduction and in situ acetylation of compounds (13), using a platinum catalyst, to give compounds of the formula (10), where X and Q are as defined as in formula (I).

Target CCR1 antagonists (15), where R^{b} is a phenyl group, which may be substituted, for example as referred to in WO01/98273) can then be prepared by reaction of epoxide (10) with a piperidine amine as shown in scheme 3, using analogues methods to those described in WO01/98273.

Compounds of formula (II) or (III) are either commercially available or may be prepared using standard procedures well known in the art.

All novel intermediates disclosed herein form a further aspect of the invention. A further aspect of the invention therefore provides a compound of formula (I) or a salt thereof

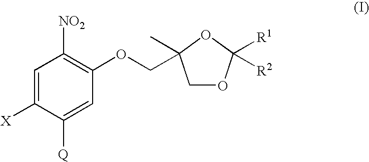

wherein Q is OH or OP where P is an alcohol-protecting group, or Q is fluorine or chlorine, X is a hydrogen atom or chlorine, and R¹ and R² together with the carbon atom to which both are attached form a 1,2 diol-protecting group.

In this aspect, the protecting group P is as defined hereinbefore. The preferred embodiments regarding X, Q, R¹ and R² referred to hereinbefore with regard to the process of the present invention apply equally to this aspect of the invention.

In an embodiment of this aspect, Q is OH or OP, or Q is fluorine. In a further embodiment of this aspect, R¹ and R² are each methyl. In a further embodiment of this aspect, X is hydrogen.

Compounds of formula (I) are capable of existing in stereoisomeric forms, and it will be understood that the invention encompasses all optical isomers of the compounds of formula (I) and mixtures thereof including racemates. In an embodiment of this aspect, R¹ and R² are each methyl and the compound of formula (I) is the R-isomer.

One embodiment relates to compounds which are
4-(5-Fluoro-2-nitro-phenoxymethyl)-2,2,4-trimethyl-[1,3]dioxolane,
4-(5-Chloro-2-nitro-phenoxymethyl)-2,2,4-trimethyl-[1,3]dioxolane,
4-Nitro-3-(2,2,4-trimethyl-[1,3]dioxolan-4-ylmethoxy)-phenol,
(S)-4-Nitro-3-(2,2,4-trimethyl-[1,3]dioxolan-4-ylmethoxy)-phenol,
(R)-4-Nitro-3-(2,2,4-trimethyl-[1,3]dioxolan-4-ylmethoxy)-phenol,
4-Amino-3-(2,2,4-trimethyl-[1,3]dioxolan-4-ylmethoxy)-phenol,
N-[4-Hydroxy-2-(2,2,4-trimethyl-[1,3]dioxolan-4-ylmethoxy)-phenyl]-acetamide,
(S)-N-[4-Hydroxy-2-(2,2,4-trimethyl-[1,3]dioxolan-4-ylmethoxy)-phenyl]-acetamide,
(R)-N-[4-Hydroxy-2-(2,2,4-trimethyl-[1,3]dioxolan-4-ylmethoxy)-phenyl]-acetamide,
Acetic acid 4-acetylamino-3-(2,2,4-trimethyl-[1,3]dioxolan-4-ylmethoxy)-phenyl ester,
4-(4-Chloro-5-fluoro-2-nitro-phenoxymethyl)-2,2,4-trimethyl-[1,3]dioxolane,
2-Chloro-4-nitro-5-(2,2,4-trimethyl-[1,3]dioxolan-4-ylmethoxy)phenol, or
N-[5-Chloro-4-hydroxy-2-(2,2,4-trimethyl-[1,3]dioxolan-4-ylmethoxy)phenyl]acetamide,
or a salt thereof.

In a further aspect, the present invention also provides a compound of formula (IV) or a salt thereof:

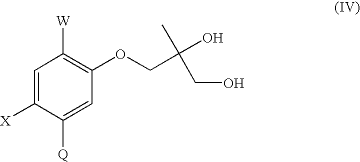

wherein W is NO₂, NH₂ or NHC(O)CH₃;
Q is OH or OP where P is an alcohol-protecting group, or Q is fluorine or chlorine;
and X is hydrogen or chlorine.

One embodiment relates to compounds which are
3-(5-Hydroxy-2-nitro-phenoxy)-2-methyl-propane-1,2-diol,
N-[2-(2,3-Dihydroxy-2-methyl-propoxy)-4-hydroxy-phenyl]-acetamide,
(S)-N-[2-(2,3-Dihydroxy-2-methyl-propoxy)-4-hydroxy-phenyl]-acetamide, or
Acetic acid 4-acetylamino-3-(2,3-dihydroxy-2-methyl-propoxy)-phenyl ester,
or a salt thereof.

In a further aspect, the present invention also provides a compound of formula (V) or a salt thereof:

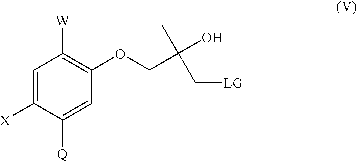

wherein W, X and Q are as defined in formula (IV) and LG is a leaving group. The leaving group is such that the compound of formula (V) can form the corresponding epoxide e.g. by treatment with a suitable base (e.g. an alkali metal base). A suitable leaving group LG is, for example, a halogen (e.g. iodine or bromine, preferably bromine) or a sulfonate ester, for example tosylate, nosylate or mesylate.

One embodiment relates to compounds which are
Acetic acid 1-(2-acetylamino-5-hydroxy-phenoxymethyl)-2-bromo-1-methyl-ethyl ester,
Acetic acid 1-(2-nitro-5-hydroxy-phenoxymethyl)-2-bromo-1-methyl-ethyl ester,
(S)-Acetic acid 1-(2-acetylamino-5-hydroxy-phenoxymethyl)-2-bromo-1-methyl-ethyl ester, or
(R)-Acetic acid 1-(2-acetylamino-5-hydroxy-phenoxymethyl)-2-bromo-1-methyl-ethyl ester, or a salt thereof.

The present invention further provides a compound of the following structure or a salt thereof:

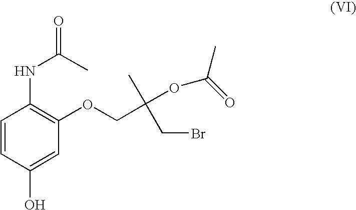

Compounds of formula (IV), (V) and (VI) may be in free or salt form. Salt forms include an alkali metal salt, for example lithium, sodium or potassium, or an alkaline earth metal salt, for example calcium or magnesium.

In a further aspect, the present invention also provides a compound of formula (VII) or a salt thereof:

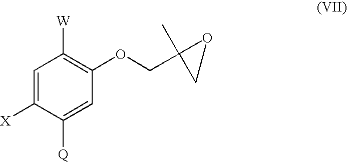

wherein W is NO₂, NH₂ or NHC(O)CH₃;
Q is OH or OP where P is an alcohol-protecting group, or Q is fluorine or chlorine;
and X is hydrogen or chlorine.

One embodiment relates to compounds which are
N-[4-Hydroxy-2-(2-methyl-oxiranylmethoxy)-phenyl]-acetamide,
(S)-N-[4-Hydroxy-2(2-methyl-oxiranylmethoxy)-phenyl]-acetamide,
(S)-Acetic acid 4-acetylamino-3-(2-methyl-oxiranylmethoxy)-phenyl ester,
3-(2-Methyl-oxiranylmethoxy)-4-nitro-phenol, or
Acetic acid 4-acetylamino-3-(2-methyl-oxiranylmethoxy)-phenyl ester, or a salt thereof.

The alcohol-protecting group P in formula (IV), (V) and (VII) is as defined hereinbefore with respect to formula (I) and formula (II). In one embodiment P is selected from C₁-C₄ alkyl groups, C₁-C₄ alkenyl groups, C₁-C₄ alkanoyl groups, C₁-C₄ alkoxycarbonyl groups, C₁-C₄ alkenyloxycarbonyl groups, aryl-C₁-C₄ alkoxycarbonyl groups, tri(C₁-C₄ alkyl)silyl and aryl-C₁-C₄ alkyl groups.

Compounds of formula (IV), (V) (VI) and (VII) are capable of existing in stereoisomeric forms, and it will be understood that the invention encompasses all optical isomers of the compounds of formula (I) and mixtures thereof including racemates. Preferred isomers are the S-enantiomer for compounds of formula (IV), and the R-enantiomer for compounds of formula (V) (where LG is halogen or sulfonate ester) and (VI).

The invention will now be further explained with reference to the following illustrative examples.

Unless otherwise specified, all starting materials and reagents were purchased from standard suppliers (Sigma Aldrich, apollo, Johnson Matthey and Fisher Scientific), and were used without further purification unless otherwise stated. The preparation and resolution of (R,S)-(2,2,4-trimethyl-1,3-dioxolane-4-yl)-methanol is known in the literature (B. Wirz, R. Barner and J. Huebscher, J. Org. Chem., 1993, 58, 3980). Reactions were carried out using standard glassware under a nitrogen atmosphere, unless otherwise stated.

NMR spectra were acquired on Varian Inova 300 mHz or 400 MHz or Bruker 300 MHz and 200 MHz spectrometers (as detailed) as solutions in suitably deuterated solvents. Nominal masses were determined either by GCMS or LCMS (as detailed). LCMS were ran on an Agilent binary 1100 HPLC with 80 Hz DAD and Multimode ES+APCI positive ion, Agilent LCMS DSL (negative ion) or a Waters 2790 HPLC equipped with 996 Photo Diode Array detector and Micromass ZMD (single quadropole mass spectrometer with Z-spray interface). GCMS data was acquired using an Agilent 6890 GC coupled to a 5973 MSD, equipped with either E1 or CI source. For CI experiments, reagent grade methane from BOC gases was used as reagent gas. Chiral HPLC was ran on an Agilent HP-1100 VWD Detector.

### EXAMPLE 1

### 4-(5-Fluoro-2-nitro-phenoxymethyl)-2,2,4-trimethyl-[1,3]dioxolane

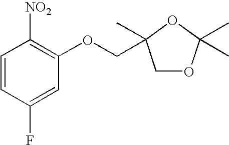

Potassium tert-butoxide (31,43 mmol; 3.64 g) was slurried with toluene (30.00 ml). (R.S)-(2,2,4-trimethyl-1,3-dioxolane-4-yl)-methanol (1.10 equiv; 34.57 mmol; 5.05 g) was diluted with toluene (10.00 ml) and added to the reaction mixture. 2,4-difluoronitrobenzene (1.00 equiv; 31.43 mmol; 5.00 g) was dissolved in a separate flask in toluene (10.00 ml) then added steadily at 0-10° C. The reaction was stirred at 0° C. for 1 h. Water (25.00 ml) was added and the two layers separated. Concentration of the organic phase in vacuo gave the title compound in 80-95% yield. Alternatively, the toluene solution can be used directly in the next stage. ¹H NMR (399.826 MHz, DMSO) δ8.03 (dd, J=9.1, 6.0 Hz, 1H), 7.39 (dd, J=11.0, 2.6 Hz, 1H), 6.99 (ddd, j=9.0, 7.9, 2.5 Hz, 1H), 4.12 (d, J=9.7 Hz, 1H), 4.04 (d, J=9.7 Hz, 1H), 4.00 (d, J=8.7 Hz, 1H), 3.74 (d, J=8.7 Hz, 1H), 1.34 (s, 3H), 1.33 (s, 3H), 1.31 (s, 3H). m/z GCMS (Cl) 314 (M+C₂H₅⁺), 286 (MH⁺), 270 (MH⁺—O), 228 (MH⁺—CH₃COCH₃).

### EXAMPLE 2

### 4-(5-Chloro-2-nitro-phenoxymethyl)-2,2,4-trimethyl-[1,3]dioxolane

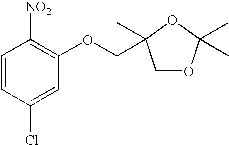

Potassium tert-butoxide (26.04 mmol; 3.01 g) was slurried with toluene (40.00 ml). (R.S)-(2,2,4-trimethyl-1,3-dioxolane-4-yl)-methanol (1.10 equiv; 28.65 mmol; 4.19 g) was diluted with toluene (20.00 ml) and added to the reaction 2,4-dichloronitrobenzene (1.00 equiv; 26.04 mmol; 5.00 g) was dissolved in a separate flask in toluene (10.00 ml) then added steadily at 0-10° C. The reaction was stirred overnight at room temperature. Water (25.00 ml) was added and the two layers separated. The organic phase was concentrated in vacuo to give the title compound in 80-95% yield. ¹H NMR (299.947 MHz, DMSO) δ 7.94 (d, J=8.8 Hz, 1H), 7.57 (d, J=1.9 Hz, 1H), 7.20 (dd, J=8.6, 2.1 Hz, 1H), 4.16 (d, J=9.8 Hz, 1H), 4.07 (d, J=9.6 Hz, 1H), 3.99 (d, J=8.8 Hz, 1H), 3.74 (d, J=8.8 Hz, 1H), 1.35 (3×s, 9H), m/z GCMS (CI) 330 (M+C₂H₅⁺), 302 (MH⁺), 286 (MH⁺—O), 244 (MH⁺—CH₃COCH₃).

### EXAMPLE 3

### 4-Nitro-3-(2,2,4-trimethyl-[1,3]dioxolan-4-ylmethoxy)-phenol

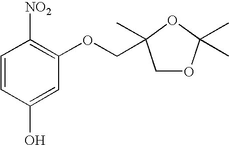

Method 1: Potassium tert-butoxide (2.74 mol; 316.64 g), N-methylpyrrolidone (300.00 ml) and toluene (700.00 ml) were added to a suitable reaction vessel at room temperature. (R,S)-(2,2,4-trimethyl-1,3-dioxolane-4-yl)-methanol (1.15 equiv; 1.46 mol; 214.02 g) in toluene (700.00 ml) was added to the reaction vessel. 3-Fluoro-4-nitrophenol (1.00 equiv; 1.27 mol; 200.00 g) was dissolved in N-methylpyrrolidone (200.00 ml) and toluene (300.00 ml) and added in a controlled manner to the reaction vessel. The reaction was heated for 1.5 h at 60-65° C. The reaction was cooled to ambient and quenched with water (1.00 l). The aqueous layer was acidified by addition of acetic acid (1.45 mol; 83.20 ml). Isopropyl acetate (2.00 l ) was added and the organic phase was separated. The product can be isolated by concentrating in vacuo to give the title compound in 95-100% yield. Alternatively, the isopropyl acetate solution can be used directly in the next stage.

Method 2: (R,s)-4-(5-fluoro-2-nitro-phenoxymethyl)-2,2,4-trimethyl-[1,3]dioxolane (1.00 equiv; 17.53 mmol; 5.00 g), as a toluene solution (30 ml) was charged to a flask containing benzyltrimethylammonium hydroxide (1.75 mmol; 771.43 μl; 732.86 mg) and 50% w/w potassium hydroxide (52.58 mmol; 4.90 ml; 5.90 g). The reaction was heated at reflux for 20 h. Water was added (35 ml) and the two phases separated. The aqueous phase was acidified with acetic acid to pH 6, then extracted with isopropyl acetate/NMP (12.5 ml/1.25 ml respectively). The organic phase was washed with water then concentrated in vacuo to give the title compound 70-90% yield. ¹H NMR (399.826 MHz, DMSO) δ7.89 (d, J=9.0 Hz, 1H), 6.61 (d, J=2.3 Hz, 1H), 6.47 (dd, J=9.2, 2.3 Hz, 1H), 4.03 (d, J=8.7 Hz, 1H), 4.00 (d, J=9.5 Hz, 1H), 3.92 (d, J=9.2 Hz, 1H), 3.74 (d, J=8.7 Hz, 1H), 1.33 (2×s, 6H), 1.32 (s, 3H). m/z LCMS (ESI+ve) 306 MNa⁺, 226 (M⁺—CH₃COCH₃).

### EXAMPLE 4

### 4-Amino-3-(2,2,4-trimethyl-[1,3]dioxolan-4-ylmethoxy)-phenol

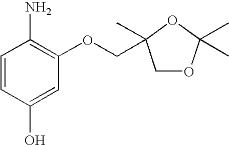

Method 1: (R,S)-4-Nitro-3-(2,2,4-trimethyl-[1,3]dioxolan-4-ylmethoxy)-phenol (4.5 g, 15.89 mmol), 5% Pd/C (0.135 g, 0.63 mmol) and ethyl acetate (67.5 ml) were charged to a hydrogenator. Hydrogenation started at ambient temperature/3-5 barg H₂. Upon completion, the reaction mixture was filtered and the solids washed with ethyl acetate (45 ml). The combined filtrates were evaporated to dryness to give the title compound in 95-100% yield.

Method 2: (R,S)-4-Nitro-3-(2,2,4-trimethyl-[1,3]dioxolan-4-ylmethoxy)-phenol (1.00 equiv; 2.39 mmol; 500.00 mg;) was added to a solution of sodium dithionite (16 mmol; 2.8 g) in water (44.07 mmol; 8.00 ml; 8.00 g;) at room temperature. The pH was adjusted to 14 using NaOH (10 M). At the end of addition the reaction was quenched by addition of 2 M HCl to pH5. The resulting precipitate was collected by filtration. The solid was dried overnight in a vacuum oven at 40° C. to give the title compound in 78% yield. ¹H NMR (299.947 MHz, DMSO) δ8.44 (s, 1H), 6.45 (d, J=8.4 Hz, 1H), 6.29 (d, J=2.3 Hz, 1H), 6.14 (dd, J=8.3, 2.4 Hz, 1H), 4.03 (m, 3H), 3.76 (m, 2H), 3.69 (d, J=9.0 Hz, 1H), 1.34 (overlapping s, 9H). m/z LCMS (ESI+ve) 276 (MNa⁺), 254 (MH⁺), 196 (MH⁺—CH₃COCH₃)

### EXAMPLE 5

### N-[4-Hydroxy-2-(2,2,4-trimethyl-[1,3]dioxolan-4-ylmethoxy)-phenyl]-acetamide

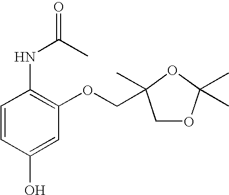

5% Pd/C (897.50 μmol; 4.51 g), (R,S)-4-nitro-3-(3,2,4-trimethyl-[1,3]dioxolan-4-ylmethoxy)-phenol (1.00 equiv; 89.75 mmol; 25.42 g) in isopropyl acetate (190 ml) and acetic acid anhydride (98.72 mmol; 9.2 ml) were charged to a suitable vessel. The mixture was hydrogenated at 20-25° C. and 4 barg H₂ overnight. The reaction was filtered and washed with water (50 ml). The isopropyl acetate was removed by distillation at atmospheric pressure (volume distilled=250 ml). The resulting solution was cooled to 20° C. and isohexane (100 ml) was added. The resulting slurry was heated to 50° C. then cooled to 20° C. over 1 h. The solid was collected by filtration and dried in a vacuum oven overnight. The title compound was isolated in 68% yield. ¹H NMR (399.819 MHz, DMSO) δ9.29 (s, 1H), 8.69 (s, 1H), 7.33 (d, J=8.5 Hz, 1H), 6.42 (d, J=2.6 Hz, 1H), 6.30 (dd, J=8.6, 2.4 Hz, 1H), 4.10 (d, J=8.7 Ha, 1H), 3.81 (d, J=9.5 Hz, 1H), 3.73 (m, 2H), 1.97 (s, 3H), 1.33 (3×s, 9H). m/z GCMS (EI) 295 (M⁺), 280 (M⁺—CH₃), 220 (M⁺—C₃H₇O₂), 125 (C₆H₇NO₂⁺).

### EXAMPLE 6

### 3-(5-Hydroxy-2-nitro-phenoxy)-2-methyl-propane-1,2-diol

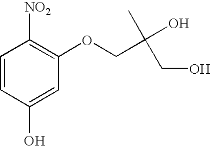

(R,S)-4-Nitro-3-(2,2,4-trimethyl-[1,3]dioxolan-4-ylmethoxy)-phenol (2.0 mmol, 0.5 g) was dissolved in ethyl acetate (5 ml) and added to a solution of 2 M HCl (0.5 ml) at room temperature. Upon completion of reaction, the two phases were separated. The organic phase was concentrated in vacuo to give the title compound in 90-95% yield. ¹H NMR (399.819 MHz, DMSO) δ10.83 (s, 1H), 7.88 (d, J=9.2 Hz, 1H), 6.56 (d, J=2.6 Hz, 1H), 6.45 (dd, J=9.0, 2.3 Hz, 1H), 3.93 (d, J=9.0 Ha, 1H), 3.78 (d, J=9.0 Hz, 1H), 3.36 (m, 2H), 1.14 (s, 3H). m/z LCMS (ESI+ve)266 (MNa⁺), 226 MH⁺—H₂O

### EXAMPLE 7

### N-[2-(2,3-Dihydroxy-2-methyl-propoxy)-4-hydroxy-phenyl]-acetamide

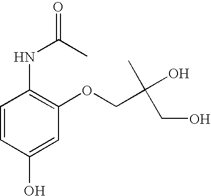

(R,S)-N-[4-hydroxy-2-(2,2,4-trimethyl-[1,3]dioxolan-4-ylmethoxy)-phenyl]-acetamide (3.8 mmol, 0.98 g) was dissolved in 2-methyl tetrahydrofuran (10 ml) at ambient temperature. Aqueous hydrochloric acid (2 M, 5 ml) was added and stirring continued at ambient temperature. At the end of reaction, ethyl acetate (10 ml) and water (10 ml) were added and the layers separated. The organic layer was washed with water (10 ml) then 20% brine (5 ml). The organic layer was evaporated to dryness in vacuo to leave the title compound in 44% yield. ¹H NMR (399.826 MHz, DMSO) δ9.22 (s, 1H), 8.87 (s, 1H), 7.55 (d, J=8.5 Hz, 1H), 6.39 (d, J=2.6 Hz, 1H), 6.28 (dd, J=8.6, 2.4 hz, 1H), 4.76 (s, 1H), 4.71 (t, J=5.6 Hz, 1H), 3.76 (d, J=9.0 Hz, 1H), 3.69 (d, J=8.6 Hz, 1H), 3.45 (dd, J=10.6, 5.5 Hz, 1H), 3.27 (m, 1H), 2.02 (s, 3H), 1.13 (s, 3H). m/z LCMS (ESI+ve) 256 (MH⁺).

### EXAMPLE 8

### Acetic acid 4-acetylamino-3-(2,3-dihydroxy-2-methyl-propoxy)-phenyl ester

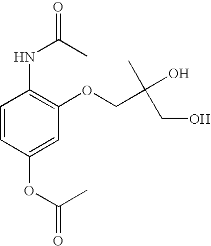

(R,S)-Acetic acid 4-acetylamino-3-(2,2,4-trimethyl-[1,3]dioxolan-4-ylmethoxy)-phenyl ester (3.2 mmol, 0.96 g) was dissolved in 2-methyl tetrahydrofuran (10 ml) at ambient temperature and aqueous hydrochloric acid (2 M, 5 ml) was added at ambient temperature. After 5 h, water (10 ml), 20% sodium chloride solution (20 ml) and toluene (10 ml) were added. The organic layer was separated and washed with water (10 ml), 20% brine (5 ml) and then evaporated to dryness in vacuo to leave the title compound in 37% yield. ¹H NMR (399.817 MHz, CDCl₃) δ8.35 (d, J=8.7 Hz, 1H), 7.88 (s, 1H), 6.72 (m, 2H), 4.12 (d, J=10.8 Hz, 1H), 3.97 (d, J=11.0 Hz, 1H), 2.92 (d, J=4.6 Hz, 1H), 2.78 (d, J=4.6 Hz, 1H), 2.27 (s, 3H), 2.20 (s, 3H), 1.48 (s, 3H). m/z LCMS (ESI+ve) 320 (M+Na⁺), 298 (MH⁺).

### EXAMPLE 9

### Acetic acid 4-acetylamino-3-(2,2,4-trimethyl-[1,3]-dioxolan-4-ylmethoxy)-phenyl ester

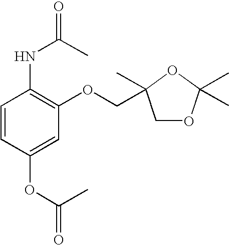

(R,S)-4-Amino-3-(2,2,4-trimethyl-[1,3]dioxolan-4-ylmethoxy)-phenol (3.5 mmol, 0.88 g) was dissolved in 2-methyl tetrahydrofuran (9 ml) and charged to the reaction flask. Triethylamine (1.45 ml) was added and the mixture cooled in ice-water. Acetyl chloride was added at a controlled rate so that the internal temperature was maintained below 15° C. The cooling bath was removed and the reaction mixture was allowed to warm to ambient temperature and stirred overnight. Water (9 ml) was added to the reaction mixture and stirring continued briefly. The layers were separated and the organic layer washed with 20% sodium chloride solution (5 ml). The organic layer was concentrated to dryness in vacuo to leave the title compound in 85% yield. ¹H NMR (399.819 MHz, DMSO) δ8.88 (s, 1H), 7.70 (d, J=8.7 Hz, 1H), 6.89 (d, J=2.6 Hz, 1H), 6.66 (dd, J=8.6, 2.4 Hz, 1H), 4.13 (d, J=8.7 Hz, 1H), 3.89 (d, J=9.5 Hz, 1H), 3.79 (d, J=9.5 Hz, 1H), 3.74 (d, J=8.7 Hz, 1H), 2.24 (s, 3H), 2.04 (s, 3H), 1.34 (m, 9H). m/z LCMS (ESI+ve) 360 (MNa⁺), 338 (MH⁺), 280 (MH⁺—CH₃COCH₃).

### EXAMPLE 10

### Acetic acid 1-(2-acetylamino-5-hydroxy-phenoxymethyl)-2-bromo-1-methyl-ethyl ester

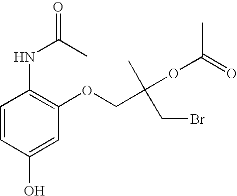

To a 50 ml 3-neck flask was added (R,S)-N-{4-hydroxy-2-[(2,2,4-trimethyl-1,3-dioxolan-4-yl)methoxy]phenyl}acetamide^{#}(1.00 equiv; 6.77 mmol; 2.00 g) and acetic acid (20.00 ml). After stirring for 10 min, 33% w/w hydrogen bromide in acetic acid (20.85 mmol; 3.60 ml; 5.11 g) was added over a period of 2 min. After 4.5 h, the reaction was quenched with sodium hydroxide (100.00 ml), and extracted with tetrahydrofuran (20.00 ml). The organic phase was separated. The aqueous phase was extracted with a further portion of tetrahydrofuran (20.00 ml). The combined organic phases were concentrated in vacuo to give the title compound in 85% yield. ¹H NMR (299.947 MHz, DMSO) δ8.80 (s, 1H), 7.27 (d, J=8.4 Hz, 1H), 6.42 (d, J=2.5 Hz, 1H), 6.33 (dd, J=8.4, 2.3 Hz, 1H), 4.19 (d, J=9.6 Hz, 1H), 4.07 (m, 3H), 2.00 (s, 3H), 1.91 (s, 3H). m/z LCMS (ESI+ve) 382 (MNa⁺), 360 (MH⁺).

^{#}(R,S)-N-{4-hydroxy-2-[(2,2,4-trimethyl-1,3-dioxolan-4-yl)methoxy]phenyl}acetamide can be substituted with (R,S)-Acetic acid 4-acetylamino-3-(2,3-dihydroxy-2-methyl-propoxy)-phenyl ester, (R,S)-N-[2-(2,3-Dihydroxy-2-methyl-propoxy)-4-hydroxy-phenyl]-acetamide or (R,S)-Acetic acid 4-acetylamino-3-(2,2,4-trimethyl-[1,3]-dioxolan-4-ylmethoxy)-phenyl ester or mixtures thereof.

### EXAMPLE 11

### N-[4-Hydroxy-2-(2-methyl-oxiranylmethoxy)-phenyl]-acetamide

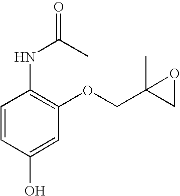

Methanol (3.00 ml) was added to (R,S)-acetic acid 1-(2-acetylamino-5-hydroxy-phenoxymethyl)-2-bromo-1-methyl-ethyl ester (0.84 mmol, 0.2 g) to give a dark brown solution. 25% w/w sodium methoxide in methanol (1.49 mmol; 340.00 μl; 321.30 mg) was added dropwise. The reaction was allowed to progress at ambient temperature. After 30 min the reaction was quenched with 10 ml of saturated ammonium chloride and 8 ml of brine. The phases were separated. The aqueous phase was washed with a further portion of ethyl acetate (10.00 ml). The organic phases were combined and washed with brine (10 ml), dried over magnesium sulfate, filtered, and concentrated under vacuum to give the title compound in 50% yield. ¹H NMR (299.947 MHz, DMSO) δ9.30 (s, 1H), 8.81 (s, 1H), 7.32 (d, J=8.6 Hz, 1H), 6.42 (d, J=2.3 Hz, 1H), 6.31 (dd, J632 8.4, 2.5 Hz, 1H), 4.06 (d, J=10.9 Hz, 1H), 3.83 (d, J=10.9 Hz, 1H), 2.83 (d, J=5.0 Hz, 1H, 2.68 (d, J=5.0 Hz, 1H), 1.99 (s, 3H), 1.37 (s, 3H). m/z LCMS (ESI+ve) 260 (MNa⁺), 238 (MH⁺), 220 (MH⁺—H₂O).

### EXAMPLE 12

### N-(2-{3-[1-(4-Chloro-benzyl)-piperidin-4-ylamino]-2-hydroxy-2-methyl-propoxy}-4-hydroxy-phenyl)-acetamide

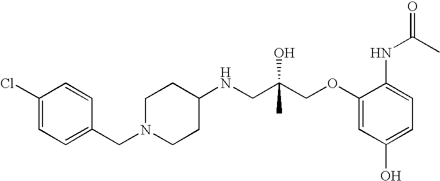

To a 25 ml 3-neck flask was added N-[4-hydroxy-2-(2-methyl-oxiranylmethoxy)-phenyl]-acetamide (1.00 equiv; 842.97 μmol; 200.00 mg). After purging with nitrogen, methanol (600.00 μl) then 1-(4-chloro-benzyl)-piperidin-4-ylamine (1.00 equiv; 845.45 μmol; 190.00 mg) in with isopropyl acetate (600.00 μl) was added. The orange solution was heated to 55° C. After stirring overnight, methanol (1.00 ml) was added. Assay of the reaction mixture against an authentic sample showed the product in 72% yield.

### EXAMPLE 13

### 4-(4-chloro-5-fluoro-2-nitro-phenoxymethyl)-2,2,4-trimethyl-[1,3]dioxolane

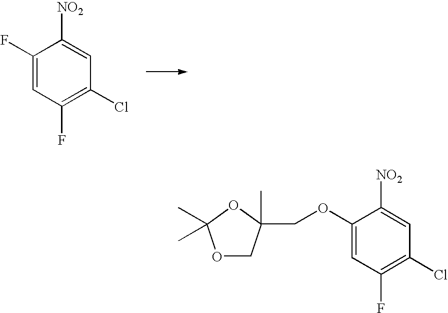

A solution of (R,S)-(2,2,4-trimethyl-[1,3]-dioxolan-4-yl)methanol (8.31 g; 56.8 mmol) in toluene (40 ml) was added slowly to a stirred slurry of potassium tert-butoxide (7.69 g, 67% w/w, 45.9 mmol) in toluene (100 ml) under an atmosphere of nitrogen. The mixture was stirred for 30 min. then cooled to −5° C. A solution of 1-chloro-2,4-difluoro-5-nitrobenzene (10.0 g; 51.7 mmol) in toluene (20 ml) was then slowly added, maintaining the temperature in the reaction vessel between −5 and 0° C. The resulting mixture was stirred for 1 h at −5 to 0° C. then analysed by HPLC. The reaction was found to be incomplete and a further portion of potassium tert-butoxide (1.77 g, 67% w/w, 10.6 mmol) was added. The mixture was stirred for a further 1 h at −5 to 0° C. after which time the reaction had reached completion. Water (70 ml) was then added slowly to the cooled (−5° C.) mixture. The mixture was allowed to warm to 20-25° C. and allowed to separate. The organic phase was washed with water (70 ml) then evaporated at low pressure at 45-50° C. to give an oil. The oil was treated with n-heptane (20 ml) then re-evaporated to again give an oil. The oil was stirred with fresh n-heptane (70 ml) for 30 min., which resulted in the precipitation of a solid. The suspension was cooled to 15° C., and then filtered. The collected solid was washed with n-heptane (20 ml) then dried in vacuo to give 4-(4-chloro-5-fluoro-2-nitro-phenoxymethyl)-2,2,4-trimethyl-[1,3]dioxolane (12.5 g; 76%). 99.5 area % purity by HPLC analysis. ¹H-NMR (200.13 MHz, CDCl₃) δ8.05 (d, J=7.4 Hz, 1H), 6,92 (d, J=10.2 Hz, 1H), 4.17 (d, J=9.0 Hz, 1H), 4.02-3.78 (m, 3H), 1.47 (s, 3H), 1.42 (s, 6H). m/z LCMS (ES⁺): 320 MH⁺), 342 (M+Na⁺)

### EXAMPLE 14

### 2-Chloro-4-nitro-5-(2,2,4-trimethyl-[1,3]dioxolan-4-ylmethoxy)phenol

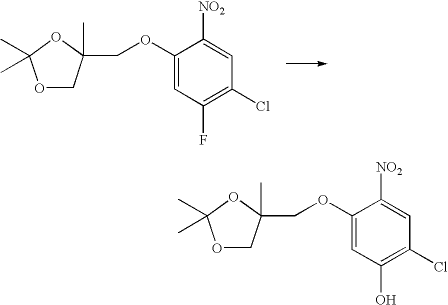

A 40% w/w aqueous solution of potassium hydroxide (7.83 g, 55.8 mmol) was added as one portion to a solution of 4-(4-chloro-5-fluoro-2nitro-phenoxymethyl)-2,2,4-trimethyl-[1,3]dioxolane (3.0 g; 9.4 mmol) in N-methyl pyrrolidine (10.5 ml) at 20-25° C. The resulting brown mixture was stirred for approximately 7 h at 20-25° C., by which time the reaction had reached completion by HPLC analysis. The pH of the reaction mixture was then adjusted to pH range 5-5.5 by the addition of glacial acetic acid (ca. 2 ml). The resulting hazy mixture was extracted with chloroform (25 ml). The organic layer was separated and evaporated under reduced pressure to give a yellow oil. n-Hexane (10 ml) was added to the oily liquid and stirred well at 20-25° C. A yellow solid precipitated and was collected by filtration and dried in vacuo at 30-40° C. to give 2-chloro-4-nitro-5-(2,2,4-trimethyl-[1,3]dioxolan-4-ylmethoxy)phenol (2.75 g; 92%), 99.5 area % purity by HPLC analysis. ¹H-NMR (300.13 MHz, CDCl₃) δ8.10 (s, 1H), 6.73 (s, 1H), 6.26 (br.s, 1H), 4.22 (d, J=9.1 Hz, 1H), 4.01-3.81 (m, 3H), 1.50 (s, 3H), 1.45 (s, 6H). m/z LCMS (ES⁻): 316 (M-H)⁻

### EXAMPLE 15

### N-[5-Chloro-4-hydroxy-2-(2,2,4-trimethyl-[1,3]dioxolan-4-ylmethoxy)phenyl]acetamide

(Method A, using sodium dithionite to reduce the nitro group)

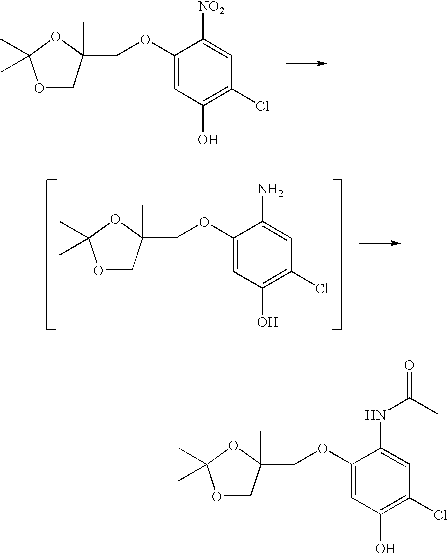

Dimethylformamide (15 ml) was charged to a reaction vessel, and cooled to 10° C. Potassium hydroxide (2.52 g; 84% w/w assay; 37.7 mmol) was then added, maintaining the temperature between 5 and 10° C. 4(4-chloro-5-fluoro-2-nitro-phenoxymethyl)-2,2,4-trimethyl-[1,3]dioxolane (1.50 g; 4.72 mmol), sodium dithionite (3.38 g; 85% w/w assay; 16.5 mmol) and water 98 ml) were added to the stirred mixture, maintaining the temperature between 5 and 10° C. The mixture was then heated to 50° c. and stirred at this temperature for 1 h. The reaction mixture was analyzed by HPLC which indicated formation of the intermediate product 4-amino-2-chloro-5-(2,2,4-trimethyl-[1,3]dioxolan-4-ylmethoxy)phenol. The mixture was cooled to 20-25° C. then filtered through a bed of hyflo supercel. The filter agent was washed with dimethylformamide (3 ml). The combined filtrates were acidified to ca. pH 6-6.5 with 20% v/v aqueous acetic acid (3 ml), then diluted with water (15 ml). The mixture was extracted with ethyl acetate 93×15 ml) and the combined ethyl acetate extracts were washed with water 92×15 ml). To the ethyl acetate solution of 4-amino-2-chloro-5-(2,2,4-trimethyl-[1,3]dioxolan-4-ylmethoxy)phenol was slowly added acetic anhydride (0.75 g; 98% w/w; 7.20 mmol) maintaining the temperature of the solution between 20 and 25° C. After the reaction had reached completion (HPLC analysis), the mixture was evaporated under reduced pressure at 45-50° C. down to a volume of approximately 10 ml. The concentrated solution was cooled to 20-25° C. and a solid precipitated. This was collected by filtration, and dried in vacuo at 40° C. to afford N-[5-chloro-4-hydroxy-2-(2,2,4-trimethyl-[1,3]dioxolan-4-ylmethoxy)phenyl] acetamide as a white solid (0.7 g; 45%), 100 area % purity by HPLC analysis. ¹H-NMR (300.13 HMz, CDCl₃) δ8.41 (s, 1H), 8.14 (br.s, 1H), 6.56 (s, 1H), 4.16 (d, J=8.8 Hz, 1H), 4.01 (d, J=9.6 Hz, 1H), 3.86 (d, J=8.8 Hz, 1H), 3.76 (d, J=9.6 Hz, 1H), 2.17 (s, 3H), 1.51 (s, 3H), 1.45 (s, 3H), 1.42 (s, 3H). m/z LCMS (ES⁺): 328 (M-H)⁻

(method B, using catalytic hydrogenation to reduce the nitro group)

4(4-Chloro-5fluoro-2nitro-phenoxymethyl)-2,2,4-trimethyl-[1,3]dioxolane (1.0 g; 3.15 mmol) was charged to a Buchi glass hydrogenation vessel followed by ethyl acetate (15 ml) and acetic acid (0.4 g). The resulting solution was inerted with nitrogen and 5% palladium on carbon (50% water wet, 140 mg) was charged to the vessel followed by additional ethyl acetate (10 ml). The mixture was then hydrogenated at 3.0 barg pressure and heated to 45-50° C. The reaction was monitored by HPLC and determined to be complete after 4 h. The mixture was cooled to 20-25° C., inerted with nitrogen, and the catalyst filtered off through a bed of hyflo supercel. The filtrate (a solution of 4-amino-2-chloro-5-(2,2,4-trimethyl-[1,3]dioxolan-4-ylmethoxy)phenol) was transferred to a clean vessel and acetic anhydride (0.48 g; 4.7 mmol) was slowly added with stirring, maintaining the temperature between 20 and 25° C. the mixture was stirred for 90 min after which time the reaction had reached completion (HPLC analysis). The solvent was evaporated under reduced pressure at 45-50° C., and n-heptane (10 ml) added to the residue. The resulting slurry was stirred for 30 min then filtered. The collected solid was washed with n-heptane (2 ml) then dried in vacuo at 40° C. to constant weight. This afforded N-[5chloro-4-hydroxy-2(2,2,4-trimethyl-[1,3]dioxolan-4-ylmethoxy)phenyl]acetamide 90.6 g; 58%), 97.99 area % purity by HPLC analysis, containing the des-chloro impurity N-[4-hydroxy-2-(2,2,4-trimethyl-[1,3]dioxolan-4-ylmethoxy)phenyl]acetamide: 1.71 area % by HPLC.

### EXAMPLE 16

### 2-Chloro-5-fluoro-4-nitrophenol (adaptation of method published in WO 03/040108)

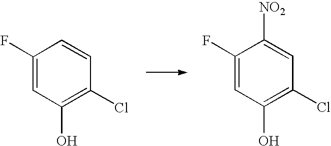

Ferric nitrate nonahydrate (14.06 g; 98% w/w; 34 mmol) was added to a solution of 2-chloro-5-fluorophenol (5.0 g; 34mmol) in ethanol (125 ml). The resulting mixture (containing suspended solid) was stirred and heated to 50-55° C. and maintained in this temperature range for 4 to 5 h, by which time the suspended solid was almost completely dissolved. Analysis by HPLC revealed complete disappearance of the starting material. The mixture was cooled to 25-30° C. and water (50 ml) was added. The mixture was then extracted with chloroform (3×25 ml) and the combined chloroform extracts washed with water 92×25 ml). The chloroform layer was evaporated under reduced pressure at 35° C. Toluene (15 ml) was added to the residue and heated to 50-55° C. and maintained within that temperature range for 10 min to give a clear solution. n-Heptane was slowly added to the solution, maintaining the temperature at 50-55° C. Crystallisation of a solid was observed during the n-heptane addition. The resulting slurry was stirred at 50-55° C. for 30 min then slowly cooled to 30-35° C. The mixture was filtered at this temperature and the collected solid washed with n-heptane (15 ml). The product was dried in vacuo at 30-35° C. to give a fluffy solid, 2-chloro-5-fluoro-4-nitrophenol (2.95 g; 45%), >98 area % purity by HPLC analysis. ¹H-NMR (200.13 MHz, CDCl₃) δ8.21 (d, J=7.4 Hz, 1H), 6.95 (d, J=11.4 Hz, 1H), 6.27 (br.s, 1H). m/z LCMS (ES⁻): 190 (M-H)⁻

### EXAMPLE 17

### 2-Chloro-4-nitro-5-(2,2,4-trimethyl-[1,3]dioxolan-4-ylmethoxy)phenol (alternative preparation starting from 2-chloro-5fluoro-4-nitrophenol)

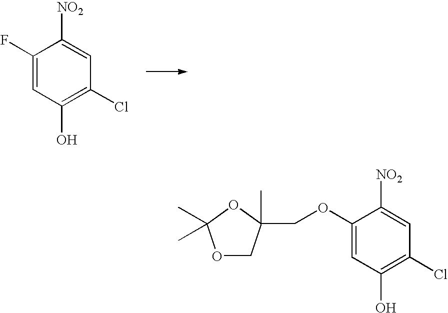

Potassium tert-butoxide (1.36 g; 95% w/w; 11.5 mmol) was added to a stirred solution of 2-chloro-5-fluoro-4-nitrophenol (1.0 g; 5.2 mmol) in acetonitrile (8 ml) under nitrogen. The resulting slurry was stirred for 10-15 min, and then a solution of racemic (2,2,4-trimethyl-[1,3]-dioxolan-4-yl)methanol (0.84 g; 5.7 mmol) in acetonitrile (2ml) was slowly added. The resulting mixture was heated to 42-50° C. and maintained at this temperature for 1-2 h after which time the reaction was complete by HPLC analysis. The mixture was cooled to 25-30° C. then filtered to remove some solid material. The solid was washed with acetonitrile (1 ml) and the combined filtrate was evaporated under reduced pressure and the residue diluted with water (20 ml) to give a 2-phase mixture. The aqueous phase was separated and extracted with chloroform (2×20 ml). The aqueous phase was then treated with dilute aqueous hydrochloric acid until pH 6 was reached. A yellow oily liquid separated out upon acidification. The 2-phase mixture was extracted with chloroform (2×20 ml). The chloroform extracts from the latter operation were combined and washed with water (10 ml). The chloroform layer was evaporated under reduced pressure at 35° C. to give 2-chloro-4-nitro-5-(2,2,4-trimethyl-[1,3]dioxolan-4-ylmethoxy)phenol (1.25 g; 76%), >98 area % purity by HPLC analysis.

Analytical data (¹H-NMR and LCMS) was consistent with the product obtained from 4-(4-chloro-5fluoro-2nitro-phenoxymethyl)-2,2,4-trimethyl-[1,3]dioxolane as described previously (Example 13).

### EXAMPLE 18

### (S)-Nitro-3-(2,2,4-trimethyl-[1,3]dioxolan-4-ylmethoxy)-phenol

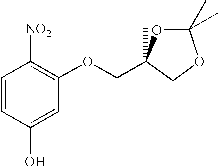

Toluene (126 ml) and N-methyl pyrrolidinone (54 ml) were added to potassium tert-butoxide (57.8 g, 2,25 equiv.). (R)-2,2,4-trimethyl-1,3-dioxolane-4-methanol 938.5 g, 1.15 equiv.) in toluene (90 ml) was added and the reaction was stirred for 30 min at room temperature. 3-Fluoro-4-nitrophenol (36 g, 1 equiv.), in N-methyl pyrrolidinone (54 ml) and toluene (36 ml) was added. The reaction was heated at 65° C. for 1.5 h. Water (180 ml) was added and the two layers were separated. Acetic acid (24.9 ml) was added to the aqueous layer and the title compound was extracted into isopropyl acetate (360 ml). The product can be isolated by concentration to dryness. Alternatively, the solution can be used directly in the next stage.

### EXAMPLE 19

### (S)-N-[4-Hydroxy-2-(2,2,4-trimethyl-[1,3]dioxolan-4-ylmethoxy)-phenyl]acetamide

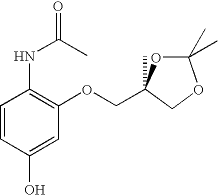

(S)-Nitro-3-(2,2,4-trimethyl-[1,3]dioxolan-4-ylmethoxy)-phenol in isopropyl acetate (32 g in 200 ml) and methanol (20 ml) were charged to Pd on C (3.1 g, 0.005 mol equiv.). The mixture was warmed to 25° C. Hydrogen was charged to the reaction at 4.25 barg. At the end of the reduction, acetic anhydride (11.2 ml) was added. The catalyst was removed by filtration. The product can be isolated by concentration to dryness. Alternatively, the solution can be used directly in the next stage.

### EXAMPLE 20

### (S)-N-[2-(2,3-Dihydroxy-2-methyl-propoxy)-4-hydroxy-phenyl]-acetamide

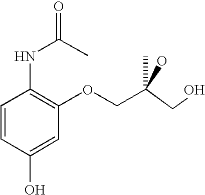

para-Toluene sulphonic acid monohydrate (2.4 g, 0.045 equiv.) was charged to (S)-N-[4-hydroxy-2-(2,2,4-trimethyl-[1,3]dioxolan-4-ylmethoxy)-phenyl]-acetamide in isopropyl acetate and methanol (82 g in 750 ml). The reaction was heated to 72° C. for 30 min. The reaction mixture was cooled to 20° C. and the methanol was removed by distillation. The title compound was collected by filtration.

### EXAMPLE 21

### (S)-Acetic acid 1-(2-acetylamino-5-hydroxy-phenoxymethyl)-2-bromo-1-methyl-ethyl ester

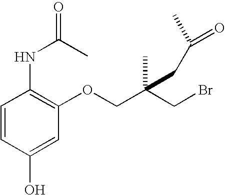

Hydrobromic acid in acetic acid (42.5 ml, 3 equiv.) was added to (s)-N-[2-(2,3-dihydroxy-2-methyl-propoxy)-4-hydroxy-phenyl]-acetamide (20 g) in acetic acid (40 ml) at 40° C. The reaction was heated at 40° C. for approximately 2 h. Isopropyl acetate (200 ml) was added followed by water. The aqueous phase was removed and the organic layer was washed sequentially with ammonium hydroxide solution and sodium sulfite solution. The product can be isolated by concentration to dryness. Alternatively, the solution can be used directly in the next stage.

### EXAMPLE 22

### (S)-Acetic acid 4-acetylamino-3-(2-methyl-oxiranylmethoxy)-phenyl ester

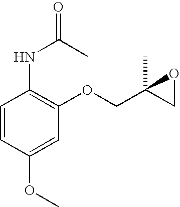

Sodium methoxide (41.2 ml, 2.3 equiv.) was added to (S)-acetic acid 1-(2-acetylamino-5-hydroxy-phenoxymethyl)-2-bromo-1methyl-ethyl ester (approx 78 mmol, 160 ml), at −10°C. After 30 min at this temperature, acetic anhydride (10 ml, 1.35 mol equiv.) was added at −5° C. This reaction was stirred for 30 min then quenched by addition of water. The two phases were separated and the organic phase was washed with sodium bicarbonate solution. The organic phase was concentrated by distillation, then diluted with heptane 940 ml). The solution was cooled to induce crystallisation, and the title compound was isolated by filtration.

### EXAMPLE 23

### (S)-N-(2-{3-[1-(4-chloro-benzyl)-piperidin-4ylamino]-2-hydroxy-2-methyl-propoxy}-4-hydroxy-phenyl)-acetamide benzoate

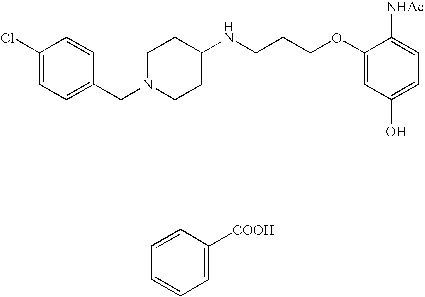

Methanol (6b 17 ml) and isopropyl acetate (8.5 ml) were charged to (S)-acetic acid 4-acetylamino-3(2-methyl-oxiranylmethoxy)-phenyl ester (10 g). The reaction mixture was heated at 40° C. and 1-(4-chloro-benzyl)-piperidin-4-ylamine in isopropyl acetate/methanol (23.9 g, 1 equiv.) was added. The reaction was heated to 55° C. for 16-24 h. Benzoic acid (3.9 g, 1 equiv.) was added and the mixture was seeded to include crystallisation. The mixture was cooled to −5° C. and the title compound was isolated by filtration. ¹H-NMR (399.824 MHz, CD₃OD) δ7.92 (m, 2H), 7.49-7.18 (m, 8H), 6.47 (d, J=2.6 Hz, 1H), 6.36 (dd, J=8.5, 2.6 Hz, 1H), 3.88 (s, 2H), 3.54 (s, 2H), 3.14 (d, J=12.3 Hz, 1H), 3.05-2.89 (m, 4H), 2.17 (t, J=11.7 Hz, 2H), 2.09 (s, 3H), 2.03 (d, J=11.7 Hz, 2H), 1.73-1.60 (m, 2H), 1.35 (s, 3H). m/z LCMS (ESI+ve) 462.20 (MH⁺).

Chiral HPLC showed that the product was enantiomerically enriched, with respect to the (S) enantiomer.

### EXAMPLE 24

### 3-Fluoro-4-nitrophenol

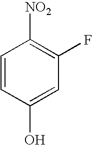

THF (400 ml) was added to ferric nitrate, nonahydrate (91 g, 223 mmol). The mixture was stirred vigorously for 10 min at room temperature. 3-Fluorophenol (50 g, 446 mmol) was dissolved in THF and heated to 42° C. The reaction was stirred overnight at this temperature, then filtered to remove the inorganic salts. The title compound can be isolated by crystallisation from toluene or by column chromatograhy. ¹H-NMR (399.822 MHz, DMSO) δ11.48 (s, 1H), 8.07 (m, 1H), 6.84-6.76 (m, 2H). ^{·}F-NMR (376.209 MHz, DMSO) δ−114.28. m/z LCMS (ESI−ve) 156.00 (M-H).

### EXAMPLE 25

### (R,S)-Acetic acid 1-(2-nitro-5-Hydroxy-phenoxymethyl)-2-bromo-1-methyl-ethyl ester

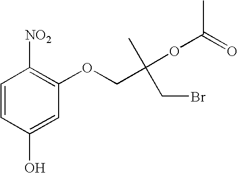

To a 100 ml 3-neck flask was added a 16.4% w/w solution of (R,S)-4-nitro-3-(2,2,4-trimethyl-[1,3]dioxolan-4-ylmethoxy)-phenol (12.2 g, 7.1 mmol) in isopropyl acetate. After stirring for 10 min at 45-50° C. 33% w/w hydrogen bromide in acetic acid (3.9 ml, 22.1 mmol) was added over a period of 2 min. After 1.5 h, the mixture was quenched with water (10 ml). After separation, the organic phase was sequentially washed with NH₄OH 1 M (20 ml) and Na₂SO₃ 12.5% w/v (20 ml). The organic solution was concentrated in vacuo to yield the title product in 95% yield. ¹H-NMR (299.947 MHz, DMSO) δ10.95 (s, 1H), 7.92 (d, J=9.0 Hz, 1H), 6.62 (s, 1H), 6.52 (d, J=9.0 Hz, 1H), 4.36 (m, 2H), 3.99 (m, 2H), 2.00 (s, 3H), 1.63 (s, 3H). m/z LCMS (ESI−ve) 347 (M-H).

### EXAMPLE 26

### 3-(2-methyl-oxiranylmethoxy)-4-nitro-phenol

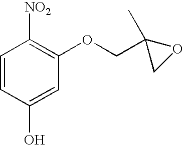

To a 50 ml 3-neck flask was added the isopropyl acetate solution of (R,S)-acetic acid 1-(2-nitro-5-hydroxy-phenoxymethyl)-2-bromo-1-methyl-ethyl ester (9.5 ml, 6.6 mmol). the mixture was cooled to −5° C. and 25% w/w sodium methoxide in methanol (3.7 ml, 16.24 mmol) was added dropwise. The reaction was allowed to progress at ambient temperature. After 30 min the reaction was quenched with water (10 ml). The biphasic mixture was separated and acetic acid (0.61 ml, 10.6 mmol) was added to the aqueous phase. The aqueous solution was extracted with isopropyl acetate (20 ml). The organic solution was concentrated in vacuo to yield the title product in 69% yield. ¹H-NMR (299.947 MHz, DMSO) δ10.90 (s, 1H), 7.91 (d, J=9.0 Ha, 1H), 6.58 (s, 1H), 6.49 (d, J=9.0 Hz, 1H), 4.14 (dd, J=10.8, 85.1 Hz, 2H), 2.80 (dd, J=5.4, 43.8 Hz, 2H), 1.40 (s, 3H). m/z LCMS (ESI+ve) 226 (MH⁺).

### EXAMPLE 27

### Acetic acid 4-acetylamino-3-(2-methyl-oxiranylmethoxy)-phenyl ester

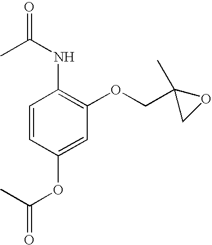

To an hydrogenation reactor were charged 3-(2-methyl-oxiranylmethoxy)-4-nitro-phenol (5g, 22.2 mmol), isopropyl acetate (50 ml), triethylamine (9.3 ml, 66.6 mmol), acetic anhydride (7.4 ml, 77.5 mmol) and 1% platinum on charcoal (22.6 μmol Pt, 1 g, 55.9% water). The mixture was stirred at 25° C. under 4 barg of hydrogen. After complete hydrogenation, the reaction mixture was filtered on buchner to remove the catalyst. The organic solution was washed with sodium carbonate and brine. The washed organic solution was concentrated in vacuo to yield the title compound in 97% yield. ¹H-NMR (299.947 MHz, DMSO) δ9.1 (s, 1H), 7.70 (d, J=8.7 Hz, 1H), 6.90 (d, J=2.4 Hz, 1H), 6.70 (dd, J632 2.4, 8.4 Hz, 1H), 4.05 (m, 2H), 2.80 (m, 2H), 2.3 (s, 3H), 2.1 (s, 3H), 1.40 (s, 3H). m/z LCMS (ESI+ve) 280.2 (MH⁺), 262.2 (MH⁺—H₂O), 220.2 (MH⁺—H₂O—CH₃CO).

### EXAMPLE 28

In addition to the racemic synthesis, the same set of reaction conditions can be used to prepare enantiomerically enriched products when (R,S)-2,2,4-trimethyl-1,3-dioxolane-4-methanol is replaced with (R)-2,2,4-trimethyl-1,3-dioxolane-4-methanol or (S)-2,2,4-trimethyl-1,3-dioxolane-4-methanol. An example with (R)-2,2,4-trimethyl-1,3-dioxolane-4-methanol is described below.

### (S)-N-[4-Hydroxy-2-(2-methyl-oxiranylmethoxy)-phenyl]-acetamide

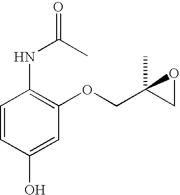

Potassium tert-butoxide (9.31 mmol; 1.04 g) and toluene (5.44 ml) were charge to a round bottomed flask under nitrogen with cooling from an ice-bath. A solution of (R)-2,2,4-trimethyl-1,3-dioxolane-4-methanol (1.08 equiv; 4.65 mmol; 680.21 mg) in toluene (1.36 ml) was added. 3-Fluoro-4-nitrophenol (1.00 equiv; 4.33 mmol; 680.00 mg) was dissolved in acetonitrile (1.70 ml) and added to the mustard slurry. The reaction was heated at 65-70° C. for 1 h. Water (6.80 ml) was added and the two layers separated. The aqueous phase was acidified with concentrated HCl (0.5 ml) to pH 5 (product oiled out during addition). Ethyl acetate (6.80 ml) was added and the two layers separated. The organic phase contained (R)-4-nitro-3-(2,2,4-trimethyl-[1,3]dioxolan-4-ylmethoxy)-phenol that was used directly in the next step.

5% Pd/C (43.42 μmol; 217.96 mg) and acetic anhydride (4.34 mmol; 410.44 μl; 443.27 mg) were charged to the organic phase and the mixture was hydrogenated at 25° C. and 5 barg overnight. The reaction was filtered and washed with water (7 ml). The organic phase was separated, washed with saturated sodium bicarbonate (3×7 ml) and concentrated in vacuo. The crude product was purified by flash chromatography using silica gel and isohexane/ethyl acetate to give 200 mg of (R)-N-[4-hydroxy-2-(2,2,4-trimethyl-[1,3]dioxolan-4-ylmethoxy)-phenyl]-acetamide.

This was dissolved in a mixture of acetic acid (50.61 mmol; 2.90 ml; 3.04 g) and 33% w/w hydrogen bromide in acetic anhydride (2.03 mmol; 350.00 μl; 497.00 mg) and heated to 35° C. After 45 min the reaction was quenched with sodium hydroxide (10.20 ml). The resulting slurry was washed into a separating funnel using ethyl acetate (10.00 ml). The two phases were separated. The aqueous was washed with a further portion of ethyl acetate (10.00 ml). The organics were combined and were washed with brine (7 ml) before being dried over magnesium sulfate, filtered, and concentrated in vacuo to give (R)-acetic acid 1-(2-acetylamino-5-hydroxy-phenoxymethyl)-2bromo-1-methyl-ethyl ester (0.2 g). this was dissolved in methanol (74.12 mmol; 3.00 ml; 2.38 g) and 25% w/w sodium methoxide in methanol (1.49 mmol; 340.00 μl; 321.30 mg) was added dropwise. After 30 min the reaction was quenched with 10 ml of saturated ammonium chloride. The two phases were separated. (To aid phase separation 8 ml of brine was added). The aqueous phase was washed with a further portion of ethyl acetate (10.00 ml). The combined organic extracts were combined, washed with brine (10 ml), dried over magnesium sulfate, filtered, and concentrated under vacuum to give the title compound in 62% yield from (R)-N-[4-hydroxy-2-(2,2,4-trimethyl-[1,3]dioxolan-4-ylmethoxy)-phenyl]-acetamide. Chiral HPLC showed that the product was enantiomerically enriched, with respect to the (S) enantiomer.

1. A process of preparing a compound of formula (I) or a salt thereof: 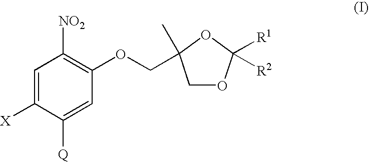
wherein Q is Oh or OP where P is an alcohol-protecting group or Q is fluorine or chlorine,
X is hydrogen or chlorine,
and R¹ and R² together with the carbon atom to which both are attached form a 1,2 diol protecting group,
which process comprises reacting a compound of formula (II) or a salt thereof 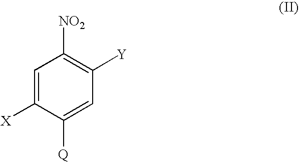
wherein Q and X are as defined in formula (I), and Y is chlorine or fluorine, with a compound of formula (III) or a salt thereof 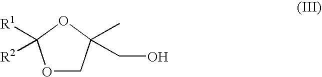
wherein r¹ and R² are as defined in formula (I), in the presence of a base.

2. A process according to claim 1, wherein R¹ and R² each independently represent hydrogen or C₁-C₆ alkyl, or R¹ and R² together with the carbon atom to which they are both attached form a C₅-C₇ cycloalkyl ring; or R¹ is hydrogen or methyl and R² is phenyl or 4-methoxyphenyl.

3. A process according to claim 1, wherein R¹ and R² are each methyl.

4. A process according to claim 1, wherein Y is fluorine.

5. A process according to claim 1, wherein Q is OH.

6. A process according to claim 1, wherein Q is fluorine.

7. A process according to claim 1, wherein X is hydrogen.

8. A process according to claim 1, wherein X is chlorine.

9. A process according to claim 1, wherein X is hydrogen, Q is Oh or OP, and Y is fluorine.

10. A process according to claim 1, wherein X is hydrogen, Q is fluorine and Y is fluorine.

11. A process according to claim 1, wherein a compound of formula (II) is reacted with a compound of formula (III) in toluene or a mixture of toluene and N-methyl pyrrolidinone.

12. A process according to claim 1, wherein Q and Y are each chlorine or Q and Y are each fluorine, and the reaction is carried out in a non-polar solvent.

13. A process according to claim 12, wherein Q and Y are each chlorine or Q and Y are each fluorine, and the reaction is carried out in toluene.

14. A process according to claim 1, wherein P is selected from C₁-C₄ alkyl groups, C₁-C₄ alkenyl groups, C₁-C₄ alkanoyl groups, C₁-C₄ alkoxycarbonyl groups, C₁-C₄ alkenyloxycarbonyl groups, aryl-C₁-C₄ alkoxycarbonyl groups, tri(C₁C₄ alkyl)silyl and aryl-C₁-C₄ alkyl groups.

15. A compound of formula (I) or a salt thereof 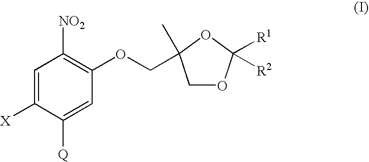
wherein Q is OH or OP where P is an alcohol protecting group, or Q is chlorine or fluorine;
X is hydrogen or chlorine;
and R¹ and R² together with the carbon atom to which both are attached form a 1,2 diol-protecting group.

16. A compound according to claim 15, wherein Q is OH or OP, or Q is fluorine.

17. A compound according to claim 15, wherein X is hydrogen.

18. A compound according to claim 15, wherein R¹ and R² are each methyl.

19. A compound which is
4-(5-Fluoro-2-nitro-phenoxymethyl)-2,2,4-trimethyl-[1,3]dioxolane,
4-(5-Chloro-2-nitro-phenoxymethyl)-2,2,4-trimethyl-[1,3]dioxolane,
4-Nitro-3-(2,2,4-trimethyl-[1,3]dioxolan-4-ylmethoxy)-phenol,
(S)-4-Nitro-3-(2,2,4-trimethyl-[1,3]dioxolan-4-ylmethoxy)-phenol,
(R)-4-Nitro-3-(2,2,4-trimethyl-[1,3]dioxolan-4-ylmethoxy)-phenol,
4-Amino-3-(2,2,4-trimethyl-[1,3]dioxolan-4-ylmethoxy)-phenol,
N-[4-Hydroxy-2-(2,2,4-trimethyl-[1,3]dioxolan-4-ylmethoxy)-phenyl]-acetamide,
(S)-N-[4-Hydroxy-2-(2,2,4-trimethyl-[1,3]dioxolan-4-ylmethoxy)-phenyl]-acetamide,
(R)-N-[4-Hydroxy-2-(2,2,4-trimethyl-[1,3]dioxolan-4-ylmethoxy)-phenyl]-acetamide,
Acetic acid 4-acetylamino-3-(2,2,4-trimethyl-[1,3]dioxolan-4-ylmethoxy)-phenyl ester,
4-(4-Chloro-5-fluoro-2-nitro-phenoxymethyl)-2,2,4-trimethyl-[1,3]dioxolane,
2-Chloro-4-nitro-5-(2,2,4-trimethyl-[1,3]dioxolan-4-ylmethoxy)phenol, or
N-[5-Chloro-4-hydroxy-2-(2,2,4-trimethyl-[1,3]dioxolan-4-ylmethoxy)phenyl]acetamide,
or a salt thereof.

20. A compound of formula (IV) or a salt thereof: 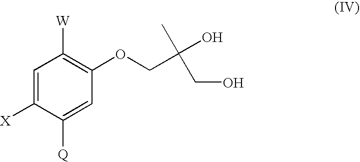
wherein W is NO₂, NH₂ or NHC(O)CH₃;
Q is OH or OP where P is an alcohol-protecting group, or Q is fluorine or chlorine;
and X is hydrogen or chlorine.

21. A compound which is
3-(5-Hydroxy-2-nitro-phenoxy)-2-methyl-propane-1,2-diol,
N-[2-(2,3-Dihydroxy-2-methyl-propoxy)-4-hydroxy-phenyl]-acetamide,
(S)-N-[2-(2,3-Dihydroxy-2-methyl-propoxy)-4-hydroxy-phenyl]-acetamide, or
Acetic acid 4-acetylamino-3-(2,3-dihydroxy-2-methyl-propoxy)-phenyl ester,
or a salt thereof.

22. A compound of formula (V) or a salt thereof, wherein 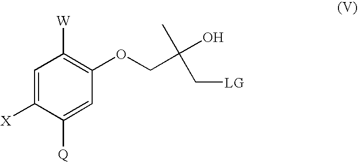
wherein W is NO₂, NH₂, NHC(O)CH₃;
Q is OH or OP where P is an alcohol-protecting group, or Q is fluorine or chlorine;
X is hydrogen or chlorine;
and LG is a leaving group.

23. A compound which is
Acetic acid 1-(2-acetylamino-5-hydroxy-phenoxymethyl)-2-bromo-1methyl-ethyl ester,
Acetic acid 1-(2-nitro-5-hydroxy-phenoxymethyl)-2-bromo-1-methyl-ethyl ester,
(S)-Acetic acid 1-(2-acetylamino-5-hydroxy-phenoxymethyl)-2-bromo-1-methyl-ethyl ester, or
(R)-Acetic acid 1-(2-acetylamino-5-hydroxy-phenoxymethyl)-2-bromo-1-methyl-ethyl ester, or a salt thereof.

24. A compound of formula (VII) or a salt thereof: 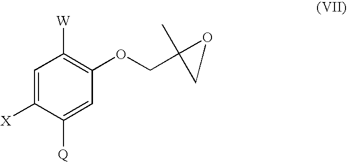
wherein W is NO₂, NH₂ or NHC(O)CH₃;
Q is OH or OP where P is an alcohol-protecting group, or Q is fluorine or chlorine;
and X is hydrogen or chlorine.

25. A compound which is
N-[4-Hydroxy-2-(2-methyl-oxiranylmethoxy)-phenyl]-acetamide,
(S)-N-[4-Hydroxy-2-(2-methyl-oxiranylmethoxy)-phenyl]-acetamide,
(S)-Acetic acid 4-acetylamino-3-(2-methyl-oxiranylmethoxy)-phenyl ester,
3-(2-Methyl-oxiranylmethoxy)-4-nitro-phenol, or
Acetic acid 4-acetylamino-3-(2-methyl-oxiranylmethoxy)-phenyl ester, or a salt thereof.

26. A compound according to claim 15, wherein P is selected from C₁C ₄ alkyl groups, C₁-C₄ alkenyl groups, C₁-C₄ alkanoyl groups, C₁-C₄ alkoxycarbonyl groups, C₁-C₄ alkenyloxycarbonyl groups, aryl-C₁C₄ alkoxycarbonyl groups, tri(C₁C₄ alkyl)silyl and aryl-C₁-C₄ alkyl groups.

27. A process for the chemoselective reduction of an aromatic nitro group in the presence of an epoxide.

28. A process for the reduction of an aromatic nitro group and in situ acetylation in the presence of an epoxide using a platinum catalyst.

29. A process for the chemoselective reduction and in situ acetylation of compounds (13), using a platinum catalyst.

30. A process for the chemoselective reduction and in situ acetylation of compounds (13), using a platinum catalyst, to give compounds of the formula (10), where X and Q are as defined according to claim 1.
